(19) United States
(12) Patent Application Publication
(10) Pub. No.: US 2013/0217085 A1
(43) Pub. Date: Aug. 22, 2013

(54) METHODS FOR IMPROVING FERMENTATION YIELD OF POLYUNSATURATED FATTY ACIDS

(71) Applicants: He Huang, Nanjing (CN); Qianqian Tong, Nanjing (CN); Lujing Ren, Nanjing (CN); Xiaojun Ji, Nanjing (CN); Aihua Xiao, Nanjing (CN); Ping Wei, Nanjing (CN); Jiangying You, Nanjing (CN); Liang Qu, Nanjing (CN); Dongping Gong, Nanjing (CN); Jiangyao Zhu, Nanjing (CN)

(72) Inventors: He Huang, Nanjing (CN); Qianqian Tong, Nanjing (CN); Lujing Ren, Nanjing (CN); Xiaojun Ji, Nanjing (CN); Aihua Xiao, Nanjing (CN); Ping Wei, Nanjing (CN); Jiangying You, Nanjing (CN); Liang Qu, Nanjing (CN); Dongping Gong, Nanjing (CN); Jiangyao Zhu, Nanjing (CN)

(73) Assignee: NANJING UNIVERSITY OF TECHNOLOGY, Nanjing ,Jiangsu (CN)

(21) Appl. No.: 13/812,496

(22) PCT Filed: Jan. 13, 2011
(86) PCT No.: PCT/CN2011/070240

§ 371 Date: Apr. 20, 2013

(30) Foreign Application Priority Data
Jul. 27, 2010 (CN) 201010237946.1

(51) Int. Cl. C12P 7/64

(52) U.S. Cl. CPC C12P 7/6427
USPC 435/134

(57) ABSTRACT

The present invention discloses a method of improving the fermentation yield of polyunsaturated fatty acids, in which Schizochytrium limacinum is used as production strain to produce polyunsaturated fatty acids (PUFA), and glycine betaine or trehalose is added to the fermentation medium. In the present invention, after Schizochytrium limacinum fermentation system is treated with exogenous glycine betaine, the yield of PUFA produced by fermentation with Schizochytrium limacinum can be greatly improved. The present invention significantly improves the yield of PUFA produced by microorganism, reduces the cost without harming the environment, and saves manpower and material resources by simple and effective regulation of fermentation, and thus the method is simple, convenient and cost-effective.

### TECHNICAL FIELD

The present invention relates to a method for enhancing the fermentation yield of polyunsaturated fatty acids, belonging to the field of biotechnology.

### BACKGROUND ART

Polyunsaturated fatty acids (PUFA), which are important constituent of biofilm of the cells and organisms, can regulate cell configuration, homeostasis, phase transition, and the permeability of the cell membrane, and also adjust the membrane-related physiological processes, so they can influence the chemical composition of the cell, the signal transmission, immunity and cold adaptation, as well as the occurrence of diseases associated therewith. PUFA can be converted into metabolites adjusting some physiological function of human. Docosahexaenoic acid (DHA), which is the main component of PUFA in cell membranes, has important physiological functions, such as enhancing memory enhancement, improving intelligence, lowering blood lipids, regulating the immune system and other effects, but also can prevent and treat cardiovascular disease , such as cancer, etc. Docosapentaenoic acid (DPA), which is a long-chain unsaturated fatty acid in the human colostrum only, is the major constituent of the human brain tissue and nerve cells and is essential to the development of the nervous system and vision, the formation of the brain, and enhancement of memory of the infant. Furthermore, DPA can promote and improve the body's immunity. DPA and DHA have a synergistic effect and show a greater therapeutic effect on type II diabetes, rheumatoid arthritis, psoriasis, asthma, ulcerative colitis and enteritis, etc., and thus have a great commercial value. In recent years, scientists have carried out a study of fermentation production of DHA with marine microorganisms. The common microorganisms include Crypthecodinium cohnii and Thraustochytrium spp., etc.Compatible solutes, which are cell metabolic intermediates, non-toxic, can adjust the osmotic pressure and prevent the radical change of the ion concentration in the cells. When the intracellular osmolality changes radically, e.g. when external osmotic pressure is elevated, the cells begin to produce or absorb several small solutes, such as trehalose, betaine and certain amino acids in order to improve the water activity within the cell to maintain the balance of osmotic pressure inside and outside of the cell, and meanwhile prevent the outflow of the cell moisture and salinity intrusion. These small solutes are “compatible solute”.

Scientists have carried out a study of fermentation production of DHA with marine microorganisms. In summary, patents published in China include the following four aspects: 1, regarding mutation screening methods of DHA-producing strain, such as “industrial application of marine fungi Schizochytrium limacinum OUC88” (200410075426.X), Ocean University of China; “a docosahexaenoic acid-producing strain and mutation screening methods and their application” (200910033493.8), Nanjing University of Technology; 2, regarding the composition of the medium, such as “a Schizochytrium limacinum and preparation of DHA oil using the same” (CN200910033869.5), Nanjing University of Technology, etc.; 3, regarding the extraction and purification of fats, such as “extraction and purification of DHA-rich fatty acid from Crypthecodinium cohnii” (200710025079.3), Nanjing University of Technology; “method of extracting unsaturated fatty acid DHA from the fermentation broth of Dinoflagellates” (CN200910159368.1), etc. Inner Mongolia Kingdomway Pharmaceutical Co., Ltd., etc.; 4, regarding DHA application, such as “maternal nutritional food (CN200610000658.8), Zhu Yan Hong etc., “preparation of ready-to-eat fish ball slice” (CN200510045178.9), Chen Yi. Currently, method of increasing the content of fatty acids by simple regulation of the fermentation has not been reported yet.

Patents concerning compatible solutes published in China generally include the following two aspects: 1, regarding extraction and preparation of compatible solutes, such as “preparation and use of an organic green feed additive” (CN200910109142.0), Shen Guangrong; “preparation of betaine” (CN00811384.X), AkzoNobel; “new method of detection and extraction of compatible solutes ectoine from neutral halophilic bacterium Halomonas salina” (CN200610135272.8), etc., Third Institute of Oceanography of the State Oceanic Administration; 2, regarding the application of compatible solutes. More is applied to improve the growth performance of the yield of animals and plants, etc., such as “a composite preparation for promoting the growth of aquatic animals to improve the meat quality and its preparation method” (CN200910307231.6), Tianjin Shengji Group Co., Ltd.; “method of improving crop yields” (95197919.1, 95197917.5 etc.), Cart limited. Etc.; one is just for microbial fermentation aspects: “a new process to improve the fermentation yield of L-glutamic acid (CN200910067618. 9), Tianjin University of Science and Technology.

Patents concerning the compatible solutes published by foreign countries include: adding compatible solutes to improve the polypeptide quantity, “cell culture performance with betaine” (10/226, 931), Brian D. Follstad, etc.; adding betaine to improve lactic acid fermentation, “Materials and methods for efficient lactic acid production” (200610109332), Shengde Zhou etc.

### SUMMARY OF THE INVENTION

The technical problems to be solved by the present invention is to provide a simple and efficient method for increasing fermentation yield of polyunsaturated fatty acids and reducing costs without harming the environment and increasing the manpower and material resources.

To solve the above technical problem, the technical solution adopted by the present invention is provided as follows:

A method of enhancing the fermentation yield of polyunsaturated fatty acids, in which Schizochytrium limacinum is used as production strain to yield polyunsaturated fatty acids, and compatible solute is added to the fermentation medium.

Wherein said compatible solute is glycine betaine or trehalose.

In the fermentation medium, the concentration of the glycine betaine is from 10 to 100 mmol/L, preferably from 10 to 70 mmol/L, and most preferably 40 mmol/L.

In the fermentation medium, the concentration of the trehalose is from 10 to 200 mmol/L, preferably from 40 to 200 mmol/L, preferably 80 mmol/L.

The yield of PUFA produced by fermentation with marine microorganism can be improved by adding a small amount of exogenous compatible solutes to the fermentation medium. Glycine betaine is the main compatible solute which is generated when Schizochytrium limacinum responds to the ambient pressure, and it is relatively cheap, about 40 rmb/kg. Trehalose is a compatible solute which is generated when the microorganism responds to ambient pressure, about 70 rmb/kg.

Under fermentation conditions, to the medium producing DHA by fermentation with Schizochytrium limacinum is added glycine betaine or trehalose to enhance the yield of PUFA. When cultured marine microorganisms are subjected to adverse fermentation conditions, exogenous compatible solute is effective. External pressure factors include high temperature, high-pressure, high-salt, hypertonia, hypotonicity and drying, etc. Schizochytrium limacinum treated with exogenous compatible solute can better cope with the changing environment of the outside world and improve the yield of DHA. The compatible solute is the stable substance in microbial cells, hence the beneficial effect of the compatible solute is long-lasting.

Beneficial effects: in the present invention, after Schizochytrium limacinum fermentation system is treated with exogenous glycine betaine, yield of PUFA produced by fermentation with Schizochytrium limacinum can be greatly improved, mass percentage of DPA in total fatty acids is increased from 11.9% to 16.2%; mass percentage of DHA in total fatty acids is increased from 44.1% to 49.8%; mass percentage of squalene is increased from 0.8% to 1.7%; and mass percentage of saturated fatty acid C14: 0 and C16: 0 in total fatty acid is significantly reduced, respectively, from 10.0% to 5.1%, and from 24.4% to 20%. DHA yield is increased from 3.9 g/L to 5.0 g/L, increased by 28%; the ratio of DHA in the total biomass (mg/g) is increased from 57 to 72; total fatty acid yield is increased from 8.8 g/L to 10 g/L .

After using appropriate amount of trehalose, DHA yield is increased from 3.9 g/L to 7.5 g/L, increased by 92%; biomass is increased from 60 g/L to 76 g/L; the ratio of DHA in total biomass (mg/g) is increased from 57 to 99; yield of total fatty acids is increased from 8.8 g/L to 16.7 g/L

The present invention significantly improves the yield of PUFA produced by microorganism and reduces cost without harming the environment and increasing manpower and material resources by simple and effective regulation of fermentation, and thus is simple, convenient and cost-effective.

### SPECIFIC MODE FOR CARRYING OUT THE INVENTION

According to the following examples, the present invention can be better understood. The person skilled in the art, however, can easily understand that the ratio of raw materials, process conditions and the results are intended to illustrate the present invention only, and should not and does not limit the invention specifically described in the claims.

The detection method of the following Examples is the same as that in the application titled “Schizochytrium limacinum and method of production of DHA oil using the same” (Application No. 200910033869.5)

### EXAMPLE 1

The strain is Schizochytrium limacinum HX-308 with deposit number CCTCC No. M209059.

Seed medium: D-glucose 40 g/L, yeast extract 2 g/L, sodium glutamate 10 g/L, MgCl₂ 3 g/L, CaCl₂·2H₂O 1 g/L, KH₂PO₄ 4 g/L, KCl 2 g/L, NaCl 15 g/L, MgSO₄·7H₂O 5 g/L, FeCl₃ 0.1 g/L. (See “A Schizochytrium limacinum and method of production of DHA oil using the same” (Application No. 200910033869.5)).

Fermentation medium: D-glucose 40 g/L, yeast extract 2 g/L, sodium glutamate 10 g/L, MgCl₂ 3 g/L, (NH₄)₂SO₄ 6 g/L, KH₂PO₄ 4 g/L, KCl 2 g/L, NaCl 15 g/L, MgSO₄·7H₂O 5 g/L, FeCl₃ 0.1 g/L. (See “A Schizochytrium limacinum and method of production of DHA oil using the same” (Application No. 200910033869.5)).

Cultural method: strain was inoculated into seed medium with inoculation amount of 5% (v/v), cultured in a 500 mL shake flask for 24 h to log phase under 170 r at 25° C., and inoculated into the fermentation medium with addition of a certain amount of glycine betaine (such as 10 mmol/L, 40 mmol/L, 70 mmol/L, 100 mmol/L) in inoculation amount of 9% (v/v) for culturing; fermentation ceased when glucose reached an amount of 0 g/L. The results are shown in Table 1.

TABLE 1

| | Additive amount of exogenous glycine | | | | | |
|---|---|---|---|---|---|---|
| Fatty acids | betaine (mM) | | | | | |
| (% total fatty acids) | 0 | 10 | 40 | 70 | 100 | 200 |
| C14:0 | 9.9 | 7.1 | 5.1 | 5.1 | 5.2 | 2.3 |
| C16:0 | 24.4 | 21.5 | 19.9 | 19.5 | 19.0 | 10.9 |
| ARA | 0.4 | 0.5 | 0.6 | 0.8 | 0.9 | ~ |
| EPA | 1.5 | 1.2 | 1.6 | 2.0 | 2.6 | 8.6 |
| DPA | 11.9 | 14.5 | 16.2 | 16.0 | 15.8 | 10.5 |
| DHA | 44.1 | 47.8 | 49.8 | 49.0 | 48.1 | 34.9 |
| Squalene | 0.8 | 1.2 | 1.7 | 2.0 | 2.3 | 1.9 |
| Others | 7.0 | 6.2 | 5.1 | 5.6 | 6.1 | 30.9 |
| DHA yield (g/l) | 3.9 | 4.2 | 5 | 4.7 | 4.3 | 2.3 |
| Biomass (g/l) | 69 | 69 | 69 | 70 | 70 | 85 |
| DHA (mg/g biomass) | 56.52 | 60.5 | 72.2 | 67.1 | 61.14 | 27 |
| Total fatty acid | 8.8 | 8.8 | 10 | 9.6 | 8.9 | 6.6 |
| yield (g/l) | | | | | | |

### EXAMPLE 2

The strain is Schizochytrium limacinum HX-308 with deposit number CCTCC No. M209059.

Seed medium: D-glucose 40 g/L, yeast extract 2 g/L, sodium glutamate 10 g/L, MgCl₂ 3 g/L, CaCl₂·2H₂O 1 g/L, KH₂PO₄ 4 g/L, KCl 2 g/L, NaCl 15 g/L, MgSO₄·7H₂O 5 g/L, FeCl₃ 0.1 g/L.

Fermentation medium: D-glucose 40 g/L, yeast extract 2 g/L, sodium glutamate 10 g/L, MgCl₂ 3 g/L, (NH₄)₂SO₄ 6 g/L, KH₂PO₄ 4 g/L, KCl 2 g/L, NaCl 15 g/L, MgSO₄·7H₂O 5 g/L, FeCl₃ 0.1 g/L.

Cultural method: the strain was inoculated into seed medium with inoculation amount of 5% (v/v), cultured in a 500 mL shake flask for 24 h to log phase under 170 r at 25° C., and inoculated into the fermentation medium with addition of a certain amount of trehalose (such as 10 mmol/L, 40 mmol/L, 80 mmol/L) in inoculation amount of 9% (v/v); fermentation ceased when glucose reached an amount of 0 g/L. The results are shown in Table 2.

TABLE 2

| | Additive amount of exogenous | | | | | | |
|---|---|---|---|---|---|---|---|
| Fatty acids | trehalose (mM) | | | | | | |
| (% total fatty acids) | 0 | 10 | 40 | 80 | 160 | 200 | 280 |
| C14:0 | 9.9 | 9.5 | 9.6 | 10.7 | 11.0 | 11.7 | 11.1 |
| C16:0 | 24.4 | 22.3 | 21.3 | 23.2 | 22.5 | 21.5 | 22.7 |
| ARA | 0.4 | 0.6 | 1.9 | 1.2 | 1.0 | ~ | 1.0 |
| EPA | 1.5 | 1.6 | 1.8 | 1.3 | 1.4 | 1.4 | 7.7 |
| DPA | 11.9 | 12.3 | 12.6 | 12.2 | 12.1 | 11.5 | 10.8 |
| DHA | 44.1 | 44.8 | 47.4 | 45 | 43.2 | 41.7 | 39.1 |
| Squalene | 0.8 | 0.8 | 0.9 | 0.9 | 0.5 | ~ | ~ |
| Others | 7.0 | 8.1 | 4.5 | 5.5 | 8.3 | 12.2 | 7.6 |
| DHA yield (g/l) | 3.9 | 4 | 4.2 | 7.5 | 7 | 6.3 | 2.2 |
| Biomass (g/l) | 69 | 70 | 74 | 76 | 78 | 81 | 76 |
| DHA (mg/g biomass) | 56.5 | 57.1 | 56.6 | 99.3 | 89.7 | 78 | 29 |
| Total fatty acid | 8.8 | 8.9 | 8.9 | 16.7 | 16.2 | 15.1 | 5.6 |
| yield (g/l) | | | | | | | |

What is claimed is:

1. A method for improving the production of polyunsaturated fatty acid, in which Schizochytrium limacinum utilizes fermentation medium to produce the polyunsaturated fatty acid, the fermentation medium containing a compatible solute.

2. The method for improving the production of polyunsaturated fatty acid according to claim 1, wherein said compatible solute is selected for a group consisting of glycine betaine and trehalose.

3. The method for improving the production of polyunsaturated fatty acid according to claim 1, wherein said Schizochytrium limacinum is Schizochytrium limacinum HX-308 (CCTCC No. M209059).

4. The method for improving the production of polyunsaturated fatty acid according to claim 2, wherein in the fermentation medium, the concentration of the glycine betaine is from 10 to 100 mmol/L.

5. The method for improving the production of polyunsaturated fatty acid according to claim 4, wherein in the fermentation medium, the concentration of the glycine betaine is from 10 to 70 mmol/L.

6. The method for improving the production of polyunsaturated fatty acid according to claim 5, wherein in the fermentation medium, the concentration of the glycine betaine is 40 mmol/L.

7. The method for improving the production of polyunsaturated fatty acid according to claim 2, wherein in the fermentation medium, the concentration of the trehalose is from 10 to 200 mmol/L.

8. The method for improving the production of polyunsaturated fatty acid according to claim 7, wherein in the fermentation medium, the concentration of the trehalose is from 40 to 200 mmol/L.

9. The method of for improving the production of polyunsaturated fatty acid according to claim 8, wherein in the fermentation medium, the concentration of the trehalose is 80 mmol/L.
